(19) United States
(12) Patent Application Publication
(10) Pub. No.: US 2018/0163328 A1
(43) Pub. Date: Jun. 14, 2018

(54) COMPRESSION KNIT PART, COMPRESSION GARMENT, DYEING METHOD FOR COMPRESSION KNIT PART, AND PRODUCTION METHOD FOR DYED COMPRESSION GARMENT

(71) Applicant: medi GmbH & Co. KG, Bayreuth (DE)

(72) Inventors: Peter Hoffeins, Bayreuth (DE); Jochen Bauer, Gefrees (DE)

(21) Appl. No.: 15/833,511

(22) Filed: Dec. 6, 2017

(30) Foreign Application Priority Data
Dec. 9, 2016 (EP) 16 203 206.4

(51) Int. Cl. D04B 1/12; A41B 11/00; D04B 1/16; D04B 1/26; D04B 21/08; D06B 3/30; D06B 11/00; A61F 13/06; D06P 3/82

(52) U.S. Cl. CPC D04B 1/126; A41B 11/00; D04B 1/16; D04B 1/26; D04B 21/08; D06B 3/30; D06P 3/52; A61F 13/066; D06P 3/8214; D10B 2401/14; D10B 2501/021; D10B 2509/028; D06B 11/0073

(57) ABSTRACT

A compression knit part composed of a compression knit including a first ground thread and a second ground thread, wherein the first ground thread is composed at least at its surface of a manufactured polyamide fiber that accepts a first dye but not a second dye, and the second ground thread is composed at least at its surface of a manufactured polyester fiber which accepts the second dye but not the first, and a weft thread configured as compression thread.

### CROSS-REFERENCE TO RELATED APPLICATIONS

The present application claims priority of EP 16 203 206.4, filed Dec. 9, 2016, the priority of this application is hereby claimed and this application is incorporated herein by reference.

### BACKGROUND OF THE INVENTION

The invention relates to a compression knit part consisting of a compression knit, a compression garment incorporating such a compression knit part, a method of dyeing such a compression knit part and a method of producing such a compression knit part.

Compression knit parts, especially in the form of compression garments and/or additions thereto/parts thereof, are already widely known in the prior art. They include a compression knit which is frequently also referred to as a compressive knit and which has the purpose to exert pressure on a specific region of the body, for example parts of the musculature, in a precise manner. A compression knit within the meaning of the present invention is accordingly designed to be worn on the body under tension and in the process exert a specific pressure on a specific region of the body. This exertion of pressure typically serves therapeutic purposes. To this end, a compression knit typically includes a weft thread configured as compression thread. Known examples of compression garments comprising at least one compression knit part are bandages and compression stockings.

Compression knits thus have specific properties, particularly in respect of their structure, which distinguish them from other knits.

Nonetheless, compression knit parts, especially in the form of and/or as part of compression garments, are in many application scenarios, for example as compression stocking and/or externally applied bandage, visible in their donned state, so their outward appearance has to meet certain requirements, for example in order to follow fashion trends and/or to be able to align the compression knit part with the user's own color preferences. In order to create patterned or otherwise color-styled compression knit parts, various in-principle approaches are already known in the prior art. For instance, the very compression knit can be produced using at least two different ground threads that are already predyed at least in part in order thus to obtain a compression knit part in a certain color or color combination. It is also known for only one of the two ground threads to be used to be predyed and to postdye the other. Ground fibers having different dyeing properties can be used for this. Yet this approach is ultimately disadvantageous because the colors for the finished product are (at least partly) fixed, and have to be sufficiently stocked to meet orders. If, then, some colors/color combinations do not sell as well as other colors/color combinations, it may have to be necessary to dispose of some finished products and resume manufacture of others.

Manufactured polyamide fibers are frequently used for compression knits. DE 1 619 575 A proposes a one-bath multicolored dyeing of textiles with regard to such manufactured polyamide fibers. Extreme changes to the dye uptake capacity of polyamides can be made by, for example, cocondensing certain substances into the polyamide-forming starting material, the concrete proposal being for cationically or anionically modified polyamides to be simultaneously dyed in two or more colors using one dyeing liquor containing side by side at least one anionic and one cationic dye.

This proposal is disadvantageous in that even with modifications to the polyamides, residual accumulations of the corresponding dyes on whichever is the other polyamide may still occur, potentially leading to staining effects which badly affect the visual impression of the finished compression knit part and/or compression garment.

### SUMMARY OF THE INVENTION

The problem addressed by the invention is therefore that of providing a one-bath multicolored dyeing method which is suitable for compression knits and improved by comparison.

The problem is solved according to the invention by providing a compression knit, a compression garment, a method of dyeing a compression knit and a method of producing a dyed compression knit part or compression garment.

The compression knit part of the invention consists of a compression knit comprising:
a first ground thread and a second ground thread, wherein the first ground thread consists at least at its surface of a manufactured polyamide fiber that accepts a first dye but not a second dye, and the second ground thread consists at least at its surface of a manufactured polyester fiber which accepts the second dye but not the first,
a weft thread configured as compression thread.

The proposal accordingly is to construct a compression knit from initially still undyed ground threads, one of which may form a patterning thread. The ground threads differ by their surface dye affinity such that every one of the ground fibers accepts a dye which is not accepted by the other ground fiber. This is accomplished by using firstly manufactured polyamide fibers (PA), but secondly also manufactured polyester fibers (PES), the preference being to use cationically dyeable polyester (often also “cationic polyester”). Due to the use of these two different manufactured fibers, which form the surface of the ground threads at least essentially, there is a difference in dyeing properties such that staining effects will no longer occur; that is, a deposition of the first dye onto the manufactured polyester fiber and/or of the second dye onto the manufactured polyamide fiber is foreclosed or at least so minimal that no visible impairments occur.

The still undyed compression knit part, then, is simple to dye as and when required in a one-bath liquor containing the two dyes by the dyeing method which the invention provides and which will be more particularly described hereinbelow. In other words, it suffices to keep a stock of undyed compression knit parts since these can also be dyed in multicolored fashion as and when required at minimal inconvenience and low water consumption, especially that is at low cost. Not merely tone-on-tone effects are possible as a result. Neither an inventory/stockholding of dyed yarns nor of already dyed compression knit parts and/or compression garments is needed. Production machines employed in knitting and/or making up need not be revamped in the event of a pattern and color change. The color styling possible with the present invention extends beyond a tone-on-tone effect (light/dark) by a long way. Since polyester, especially cationically dyeable polyester is used, the dyeing temperatures may be kept low, especially below 97° C., while the dyeing method can similarly be kept cost-effective owing to the possibility of one-bath dyeing.

In this context, the compression knit part need not necessarily form a compression garment and/or the part of a compression garment, instead it is also perfectly conceivable for the compression knit part to form and/or belong to an accessory to a compression garment, that also accessories may be aligned in a color-compatible manner. The possible one-bath dyeing additionally reduces the necessary resources (water/energy/labor), resulting in low dyeing costs coupled with high levels of color fastness.

It may at this juncture be pointed out once more that a compression knit, or a compressive knit, herein is to be understood as meaning a knit which on application to a body region exerts a specific, especially therapeutically acting pressure thereon in a precise manner. It is for example the case with compression stockings as compression garments and/or with compression knit parts forming the compression garment that there are for example certain standardized compression classes describing the compression effect on body regions in a corresponding manner. When, therefore, the compression knit part, for example as a compression garment, forms a compression stocking, the latter may be assigned a certain compression class (CCL) which typically corresponds to a pressure value at the ankle.

A particularly preferred further development of the present invention provides that the compression thread comprises a thread core, especially of elastane, overwrapped with the same manufactured fiber as one of the ground threads, especially the polyamide ground fiber of the first ground thread. In this case, therefore, the surface of the compression thread also consists of one of the manufactured fibers, so the compression thread becomes dyed as well in a corresponding manner, i.e., with the first dye when manufactured polyamide fiber is used to overwrap the compression thread and with the second dye when manufactured polyester fiber is used for overwrapping. This may be used to ensure that ultimately only two colors occur in the compression knit.

In a particularly advantageous embodiment, the compression thread has a different dye affinity from the ground thread including the same manufactured fiber, in respect of the corresponding dye, to create on dyeing a tone-on-tone effect with regard to the corresponding dye. The presence of the compression thread in compression knits can accordingly be exploited to obtain an additional degree of design freedom, especially that is to obtain a tone-on-tone variation with regard to at least one of the colors. For instance, when the compression thread displays polyamide at its surface, a tone-on-tone effect, i.e., different brightnesses of the color, is obtainable with regard to the first dye, which holds correspondingly for the manufactured polyester fiber and the second dye when the compression thread displays manufactured polyester fibers as overwrap.

Specifically, a possible embodiment provides that the different dye affinity is realized by a different surface structure of the compression thread as compared with the ground thread including the same manufactured fiber, especially via a differing number of overwraps of a particular thread core with the corresponding manufactured fiber and/or via different pre-tensions of the threads. Specifically, therefore, the differing dye affinity may be tied to different surface structures of the compression thread and of the ground thread using the same manufactured fiber, for example by varying the number of overwraps of the thread core between the ground thread and the compression thread. The differing adherence of the corresponding dye accordingly creates overall an additional degree of design freedom which can be used to realize further visually interesting patternings and colorings.

It may additionally be noted once more at this juncture that different color impressions are also attainable by varying the geometry as between the compression thread dyeable with the same dye as one of the ground threads and the corresponding ground thread. It may thus be provided for example that the compression thread and a ground thread dyeable with the same dye as the compression thread differ in geometry, especially cross section. Changing the thickness of the weft thread for example can lead to a visually different, desired coloring effect.

It may be noted in this context that it is self-evidently also conceivable to establish an identical dye affinity between the compression thread and the corresponding ground thread with regard to the corresponding dye if it is desired to create a two-tone effect without tone-on-tone effects.

A structure involving an overwrapped core need not be present for the compression thread alone, but can also be realized for the ground threads. It may thus be provided that at least one of the ground threads includes a thread core, especially of elastane, overwrapped with the corresponding manufactured fiber.

One of the ground threads may conveniently be a pattern-conferring patterning thread which lies in front of the other ground thread on a face side of the compression knit. Hence it is the patterning thread which, as will be known in principle, mainly determines the patterning. A specific implementation may provide for example that the ground thread forming the patterning thread and the other ground thread are knitted in a plain construction and in a rib construction, respectively. It will be appreciated that various possibilities are conceivable in this regard with respect to the desired pattern in order to implement specific design mandates, in which case the knitting variations known in principle from the prior art may be employed.

A compression garment of the invention comprises, at least, a compression knit part of the type according to the invention, while especially the compression garment, preferably a compression stocking and/or a bandage, may also be formed by the compression knit part. It will be appreciated that the remarks regarding the compression knit part continue to hold analogously for the compression garment.

The invention further relates to a method of dyeing a compression knit part of the invention in a one-bath dyeing process, the method comprising the steps of:
pretreating the compression knit part,
admixing the first dye for the manufactured polyamide fibers into an employed liquor and dyeing the compression knit part at one or more than one first temperature,
awaiting the end of a first dyeing time span, admixing the second dye in a color other than that of the first dye to the liquor and heating up the liquor,
dyeing at one or more than one second temperature which is higher than the first temperature but not more than 97° C. in a second dyeing time span, and
aftertreating the compression knit part,
wherein a dye-separating agent is used to separate the first and second dyes.

The method of the present invention thus initially involves a step of pretreating the compression knit part in a manner which in essence corresponds to the basically known, customary pretreatment in polyamide dyeing. Especially the employed liquor, into which the compression knit part to be dyed has been immersed, is here already admixed with a wetting agent which can act on the compression knit part for a certain time span, for example 20 to 30 minutes. The aftertreatment may likewise be carried out as with the customary, basically known polyamide-dyeing processes and comprises for example the step of rinsing and drying the compression knit part.

The dye-separating agent, which keeps the two dyes separate, is conveniently already admixed into the liquor before admixture of the first dye, especially together with dyeing auxiliaries used as customary. Before the first dye, which acts on the manufactured polyamide fibers, is admixed, the liquor pH is customarily aligned in an appropriate manner, especially by admixing an acid donor, so a pH of about three becomes established, especially with attainment of the final temperature. This pH may be maintained throughout the entire dyeing process.

Initially, then, the first dye acting on the manufactured polyamide fiber is admixed into the dyebath while typically one or more than one first temperature is maintained and/or established, while especially even during the dyeing process with the first dye, i.e., the first dyeing time span, heating may already be effected up to the one or more than one second temperature or intermediate temperature. At the end of the first dyeing time span the second dye, which acts on the manufactured polyester fiber, is admixed. Since multicolored dyeing is to take place, the color produced by the first dye differs from that of the second dye. In the second dyeing time span, dyeing takes place at temperatures of at most 97° C., i.e., without superatmospheric pressure. Here the first temperature may be for example in an interval of 50 to 80° C. and the second temperature for example in an interval of 80 to 97° C.

As will be known in principle, it is advantageous for the compression knit part to be agitated in the liquor during the dyeing time spans in order to promote incipient dyeing.

Altogether, as already explained, what the dyeing method of the invention provides is in effect an on-demand way to take finished compression knit parts and/or compression garments from stock and dye them up cost-effectively in the desired colors in multicolor fashion. All this while low dyeing temperatures may be used and only a single dyebath is required, reducing the necessary resources (water/energy/labor). It will be appreciated that in other respects as well the remarks regarding the compression knit part continue to apply.

In one possible specific embodiment of the dyeing method, the first dye used is an anionic dye for dyeing the manufactured polyamide fibers and the second dye used is a cationic dye for dyeing the cationically dyeable manufactured polyester fibers. The direct dyes used are accordingly acid dyes and basic dyes that are known in principle and are freely available. Staining effects are nonetheless avoided by virtue of the manufactured fiber structures being entirely different.

An advantageous embodiment of the dyeing method according to the invention provides that the dye-separating agent used is a leveling agent. A useful leveling agent, especially with the use of anionic and cationic dyes (i.e., acid dyes and basic dyes), is the product which Rudolf GmbH, Geretsried (Germany) markets under the brandname “Rucogal CDP”. This is a specialty product based on a composition prepared from fatty alcohol ethoxylate, fatty acid ester, alkylaryl-sulfonate and alkylamine ethoxylate, anionically active, which has a separating effect on the dyes used for one-bath dyeing with basic dyes and acid dyes, and moreover offers a very good leveling effect for both the manufactured fibers. Tests have shown that, surprisingly, this leveling agent also acts as an excellent dye-separating agent and in essence prevents any interaction between the first and second dyes.

The invention lastly also provides a production method for a dyed compression knit part, including in particular a compression garment formed by the compression knit part, said method comprising the steps of:
providing and storing an undyed compression knit part of the invention type, and
receiving a color request describing two colors to be used and dyeing the compression knit part as per the color request in a dyeing method of the invention.

According to the invention, therefore, compression knit parts in various colors and color combinations do not have to be permanently kept in stock just in case, instead the still undyed compression knit parts can be kept in stock until it is known in which color combinations—described by the color request
they are required. It is then possible to meet the color request by employing the dyeing method of the invention to cost-effectively and conveniently produce—in high color fastness
a correspondingly dyed compression knit part and for example supply it to a customer. Altogether, in this way, distinctly fewer compression knit parts have to be kept in stock and there is distinctly less surplus merchandise in color combinations which, as will only turn out later, are less in demand.

The various features of novelty which characterize the invention are pointed out with particularity in the claims annexed to and forming a part of the disclosure. For a better understanding of the invention, its operating advantages, specific objects attained by its use, reference should be had to the drawings and descriptive matter in which there are illustrated and described preferred embodiments of the invention.

### BRIEF DESCRIPTION OF THE DRAWING

In the drawing:

FIG. 1 shows a basic sketch of a compression knit part according to the invention,

FIG. 2 shows a first view of the compression knit of the compression knit part of FIG. 1,

FIG. 3 shows a second view of the compression knit of the compression knit part of FIG. 1,

FIG. 4 shows a dyeing curve of a dyeing method according to the invention, and

FIG. 5 shows a flowchart for the production method of the invention.

### DETAILED DESCRIPTION OF THE INVENTION

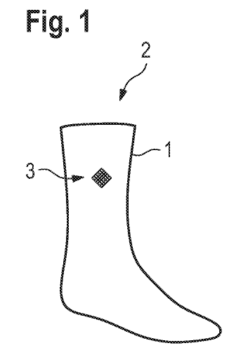

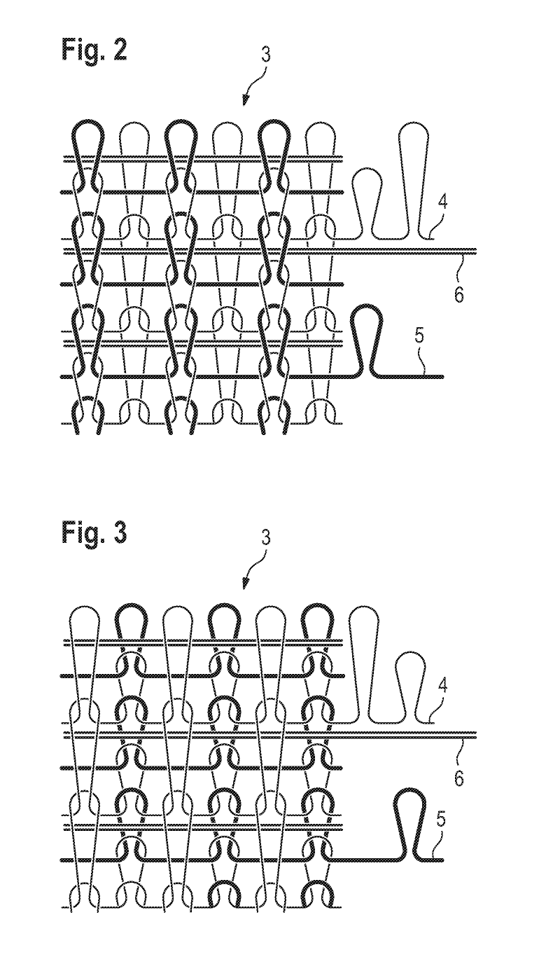

FIG. 1 shows a basic sketch of a compression knit part 1 according to the invention, which here forms a compression garment in the form of a compression stocking. The compression stocking 2 consists of the employed compression knit 3, merely suggested in FIG. 1 and more particularly depicted in FIGS. 2 and 3, and thereby develops a specific, intentional compressive effect on the covered regions of the body, particularly for therapeutic purposes. The compression stocking is more particularly assigned one of the familiar, standardized, four compression classes.

FIGS. 2 and 3 show the compression knit 3 in greater detail, the right side of the fabric in each case, FIG. 2 being a view from the viewing direction, i.e., a view of the face side, FIG. 3 being a view of the inside surface of compression stocking 2.

As FIG. 2 shows, the compression knit 3 comprises three types of threads, namely a first ground thread 4, a second ground thread 5, which forms a patterning thread, and a compression thread 6, which are extended on the right-hand side beyond the section of compression knit 3 to clarify their course. Plainly, the ground thread 5 forming the patterning thread is shown by FIG. 2 to lie on the outside in the direction of the face side, i.e., particularly above the compression threads 6 incorporated as weft threads. The ground thread 4 is located behind the compression thread 6. Accordingly, cf. FIG. 3, it is apparent when viewed from the inside surface that the ground thread 5 lies behind the compression threads 6, i.e., on the outside.

The first ground thread 4 and the second ground thread 5 have different constructions here. While both include an elastane core as thread core, the ground thread 4 is overwrapped by a manufactured polyamide fiber and the ground thread 5 is overwrapped by cationically dyeable polyester. Hence the ground threads 4 and 5 have different properties in respect of dyeability. To wit, the ground thread 4 is by virtue of the manufactured polyamide fiber solely dyeable by anionic dyes (acid dyes) as first dye, not accepting cationic dyes as second dye. By contrast, the ground thread 5 is solely dyeable by cationic dyes as second dyes on account of its surface consisting of manufactured cationically dyeable polyester fiber. The latter in turn does not accept anionic dyes.

Here the compression thread 6 is also integrated in this dyeability concept. Since the compression thread 6 itself consists of elastane overwrapped with manufactured polyamide fiber, it accordingly also accepts anionic dyes, hence the first dyes. In the present working example, the compression thread 6 and the ground thread 4 also differ in their surface structures, which is accomplished via the number of overwraps with the manufactured polyamide fiber and/or different pre-tensions. This creates a tone-on-tone effect on dyeing with the first dye, since the compression thread 6 and the ground thread 4 differ in the degree to which they accept the first dye. Working examples are also conceivable where the compression thread 6 and the ground thread 4 are provided an identical surface structure.

In a dyeing method according to the invention, the compression knit part is dyed by using a first dye and a second dye, for which dyes having different colors are used. In said method, an in-principle known step of preparation is followed by a step of one-bath dyeing absolutely without superatmospheric pressure, which is followed by the in-principle known postprocessing step.

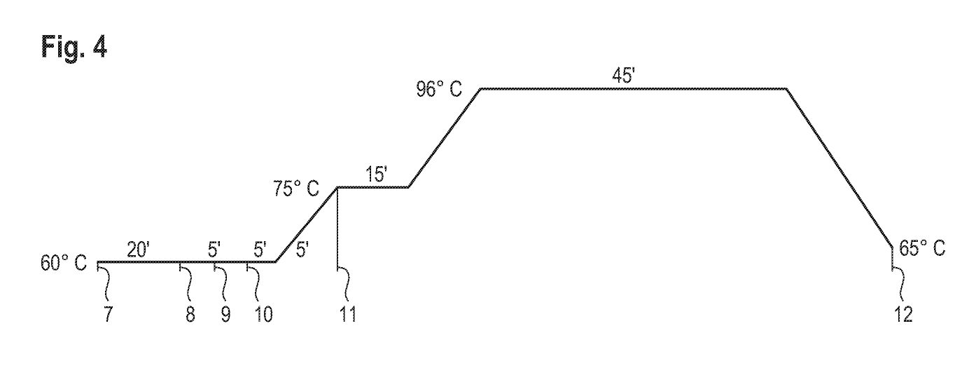

FIG. 4 shows a dyeing curve for a working example of the dyeing method according to the invention.

There the actual dyeing process, involving the use of an appropriate liquor, starts at a time 7, where the liquor, which already contains the compression knit part 1, is admixed with a wetting agent or the prepared liquor containing the wetting agent is entered with the compression knit part. The liquor temperature is initially 60° C. The compression knit part is exposed to the wetting agent for 20 minutes before at a time 8 in-principle known dyeing auxiliaries are added, but additionally also a dye-separating agent. The dye-separating agent used here is a specific leveling agent, especially the abovementioned Rucogal CDP. At a time 9 there then follows the admixture of acid donors to establish the liquor pH. These acid donors are effective in causing acid to be released over time with increasing temperature, so a pH of 3 is reached on reaching the end temperature of in this case 96° C.

A time 10 then finally sees the admixture of the first dye for the manufactured polyamide fiber. The temperature of 60° C. is additionally maintained for five minutes before heating to a temperature of 75° C. in the course of a further five minutes. Throughout the entire period, the compression knit part 1 is agitated in the liquor. The period between times 10 and 11 marks a first dyeing time span. The first dye in the form of an anionic dye (acid dye) will exhaust onto the manufactured polyamide fiber even at just 60° C. and a slightly acidic pH.

Time 11 is then the time for adding the second dye for the manufactured cationic polyester fiber, so the anionic first dye and the cationic second dye will then both be present in the liquor, yet there will not be any significant interactions, since this is prevented by the dye-separating agent. In the course of the second dyeing time span then starting, the temperature is initially maintained at 75° C. for 15 minutes before it is raised to 96° C., following which the second dyeing time span is continued for 45 minutes during which the compression knit part continues to be agitated in the liquor. Since the second temperature remains below 97° C. throughout the second dyeing time span, dyeing accordingly takes place without superatmospheric pressure. The cationic second dye requires a lower pH, which occurs at just 75° C. as a result of acid being released by the acid donor, so the exhaustion of the second dye onto the manufactured polyester fiber commences. Over time, as the temperature increases, more acid is released by the acid donor, gradually raising the affinity of the dyes until the final pH of three is reached.

After a cooling process, the time 12 marks the start for the abovementioned after¬treatment of the compression knit part 1.

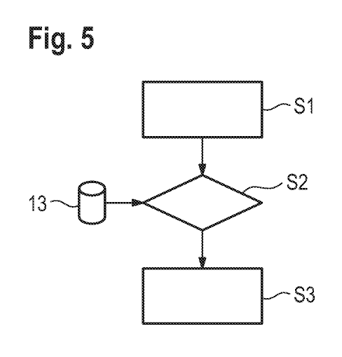

FIG. 5 shows a flowchart for a working example of a method for producing a dyed compression knit part 1. A step S1 comprises providing and inventoring the still undyed compression knit part 1. A step S2 comprises checking whether a color request 13—describing colors to be used in dyeing—has already been received for the compression knit part. If so, the compression knit part 1 is dyed in step S3 in accordance with the color request 13 in the manner described with regard to the dyeing method.

While specific embodiments of the invention have been shown and described in detail to illustrate the inventive principles, it will be understood that the invention may be embodied otherwise without departing from such principles.

We claim:

1. A compression knit part made of a compression knit comprising:
a first ground thread and a second ground thread, wherein the first ground thread is composed at least at its surface of a manufactured polyamide fiber that accepts a first dye but not a second dye, and the second ground thread is composed at least at its surface of a manufactured polyester fiber which accepts the second dye but not the first dye; and
a weft thread configured as compression thread.

2. The compression knit part according to claim 1, wherein the threads are still undyed.

3. The compression knit part according to claim 1, wherein the compression thread comprises a thread core, especially of elastane, overwrapped with the same manufactured fiber as one of the ground threads, especially the polyamide ground fiber of the first ground thread.

4. The compression knit part according to claim 3, wherein the compression thread has a different dye affinity from the ground thread including the same manufactured fiber, in respect of the corresponding dye, to create on dyeing a tone-on-tone effect with regard to the corresponding dye.

5. The compression knit part according to claim 4, wherein the different dye affinity is realized by a different surface structure of the compression thread as compared with the ground thread including the same manufactured fiber.

6. The compression knit part according to claim 1, wherein at least one of the ground threads includes a thread core, especially of elastane, overwrapped with the corresponding manufactured fiber, and/or in that the manufactured polyester fiber is composed of cationically dyeable polyester.

7. The compression knit part according to claim 1, wherein one of the ground threads is a pattern-conferring patterning thread which lies in front of the other ground thread on a face side of the compression knit.

8. The compression knit part according to claim 7, wherein the ground thread forming the patterning thread and the other ground thread are knitted in a plain construction and in a rib construction, respectively.

9. The compression garment comprising a compression knit part according to claim 1.

10. A method of dyeing a compression knit part according to claim 1 in a one-bath dyeing process, comprising the steps of:
pretreating the compression knit part,
admixing the first dye for the manufactured polyamide fibers into an employed liquor and dyeing the compression knit part at one or more than one first temperature,
awaiting the end of a first dyeing time span, admixing the second dye in a color other than that of the first dye to the liquor and heating up the liquor,
dyeing at one or more than one second temperature which is higher than the first temperature but not more than 97° C. in a second dyeing time span, and
aftertreating the compression knit part,
wherein a dye-separating agent is used to separate the first and second dyes.

11. The method according to claim 10, wherein the first dye used is an anionic dye for dyeing the manufactured polyamide fibers, the second dye used is a cationic dye for dyeing the cationically dyeable manufactured polyester fibers, and/or the dye-separating agent used is a leveling agent.

12. A method of producing a dyed compression knit part, comprising the steps of:
providing and storing an undyed compression knit part according to claim 1; and
receiving a color request describing two colors to be used and dyeing the compression knit part as per the color request in a method including the steps of:
pretreating the compression knit part,
admixing the first dye for the manufactured polyamide fibers into an employed liquor and dyeing the compression knit part at one or more than one first temperature,
awaiting the end of a first dyeing time span, admixing the second dye in a color other than that of the first dye to the liquor and heating up the liquor,
dyeing at one or more than one second temperature which is higher than the first temperature but not more than 97° C. in a second dyeing time span, and
aftertreating the compression knit part,
wherein a dye-separating agent is used to separate the first and second dyes.
